(19) United States
(12) Patent Application Publication
(10) Pub. No.: US 2009/0215993 A1
(43) Pub. Date: Aug. 27, 2009

(54) Mycophenolic acid immunogens and antibodies

(76) Inventors: Mitali Ghoshal, Fishers, IN (US); Raymond Hui, Indianapolis, IN (US); Shaker Rashid, Fishers, IN (US)

Correspondence Address: ROCHE DIAGNOSTICS OPERATIONS INC., 9115 Hague Road, Indianapolis, IN, 46250-0457, US

(21) Appl. No.: 12/257,704

(22) Filed: Oct. 24, 2008

Related U.S. Application Data
(60) Provisional application No. 60/987,840, filed on Nov. 14, 2007.

(51) Int. Cl. C07K 16/44; C07K 14/765; C07D 405/12; C07K 14/47

(52) U.S. Cl. 530/387.5; 530/363; 548/525; 530/395; 530/389.8

(57) ABSTRACT

Described are immunogenic compounds useful for generation of antibodies which are highly specific for mycophenolic acid which do not react or have substantially no cross-reactivity with the glucuronide metabolites of mycophenolic acid, especially the acyl-glucuronide metabolite.

### RELATED APPLICATIONS

This application claims benefit under 35 USC §119(e) to U.S. provisional application 60/987,840 filed Nov. 14, 2007.

### FIELD OF THE INVENTION

The invention relates to immunogenic compounds useful for generation of antibodies which are highly specific for mycophcnolic acid (mycophenolate) and which have substantially no cross-reactivity with the glucuronide metabolites of mycophenolic acid, especially the acyl-glucuronide metabolite.

### BACKGROUND

Current immunoassay methods for determining mycophenolic acid utilize antibodies derived from immunogens in which mycophenolic acid is derivatized at the carboxylic acid of mycophenolic acid (for example, the EMIT assay available from Dade-Behring) or the reduced mycophenolic acid carboxylic acid (i.e., “mycophenolic acid alcohol” used in the CEDIA assay from Microgenics).

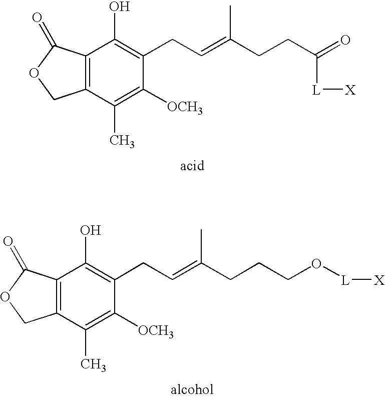

Because the mycophenolic acid molecule is linked to carrier X at the carboxylic acid or the reduced carboxylic acid, this end of the molecule is masked from the immune system and therefore cannot stimulate formation of antibody paratopes corresponding to epitopes at this position in an immunogenic response. These known inmuunoassays suffer from undesirable high cross-reactivity to the acyl-glucuronide metabolite.

HPLC-UV and LC/MS assays for mycophenolic acid are also well-known. Roche has developed and launched a non-competitive enzyme inhibition assay for mycophenolic acid employing a recombinant inosine monophosphate dehydrogenase (IMPDH). Roche also has disclosed 4′- and 5′-mycophenolic acid derivatives in the preparation of drug conjugates for a non-competitive enzyme inhibition immunoassay format which employs antibodies to the drug and IMPDH. However, immunogens prepared from 4′- and 5′-mycophenolic acid derivatives are not disclosed in the prior art.

### SUMMARY OF THE INVENTION

It is against the above background that the present invention provides certain unobvious advantages and advancements over the prior art. In particular, the inventor has recognized a need for improvements in mycophenolic acid immunogens useful for generation of antibodies having improved specificity towards mycophenolic acid parent drug, especially over glucuronide metabolites of mycophenolic acid.

The invention describes novel immunogens in which the mycophenolic acid carboxylic acid moiety is free and non-occluded. The carboxylic acid moiety is not masked as in the prior art immunogens and is therefore fully available to function as an epitope. This; is accomplished by linkage of the mycophenolic acid through different positions of the alkyl side chain, namely the 4′ and 5′ positions as shown in the following structures:

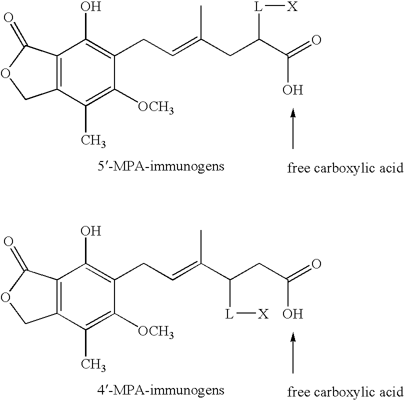

wherein L is a linker comprising from. 1 to 40 carbon atoms arranged in a straight chain or a branched chain, saturated or unsaturated, and containing up to two ring structures and 0-20 heteroatoms, with the proviso that not more than two heteroatoms may be linked in sequence and X is selected from the group consisting of polypeptides, polysaccharides and synthetic polymers.

### BRIEF DESCRIPTION OF THE DRAWINGS

FIG. 1 is a schematic showing synthesis of mycophenolic acid 5′-substituted aromatic NHS ester (10), N-{2-[(E)-3-carboxy-7-(4-hydroxy-6-methoxy-7-methyl-3-oxo-1,3-dihydro-isobenzofuran-5-yl)-5-methyl-hept-5-enoylamino]-ethyl}-terephthalamic acid 2,5-dioxo-pyrrolidin-1-yl ester.

FIG. 2 is a schematic showing synthesis of the KLH conjugate (11) and BSA conjugate (12) of mycophenolic acid 5′-substituted aromatic NHS ester (10).

FIG. 3 is a schematic showing synthesis of 5′-position maleimido-linked mycophenolic acid (4), (E)-2-({2-[3-(2,5-dioxo-2,5-dihydro-pyrrol-1-yl)-propionylamino]-ethylcarbamoyl}-methyl)-6-(4-hydroxy-6-methoxy-7-methyl-3-oxo-1,3-dihydro-isobenzofuran-5-yl)-4-methyl-hex-4-enoic acid.

FIG. 4 is a schematic showing synthesis of 5′-position linked mycophenolic acid-succinimidyl-sulfanylpropionyl-BSA conjugate (15).

FIG. 5 is a schematic showing synthesis of 5′-position linked mycophenolic acid succinimidyl-sulfanylbutyrimidoyl-KLH (16).

FIG. 6 is a schematic showing synthesis of 4′-position linked KLH conjugate (19); and BSA conjugate (20); of 4′-(2,5-dioxo-pyrrolidinyl-1-yl-oxocarbonyl-4″-phenyl-1″-carbonylamino-ethyl-2-aminocarbonylthethyl)-MPA(18).

### DETAILED DESCRIPTION OF THE INVENTION

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and, defining the present-invention it is, noted that the term “substantially” is utilized herein to represent the inherent degree of uncertainty that may be-attributed to any quantitative comparison, value, measurement, or other representation. The term “substantially” is also utilized herein to represent the degree by which a quantitative representation may vary from a stated reference without resulting in a change in the basic function of the subject matter at issue.

The invention provides immunogens for generation of highly specific antibodies to mycophenolic acid which do not react with the glucuronide metabolites of mycophenolic acid, especially the acyl-glucuronide metabolite as shown below:

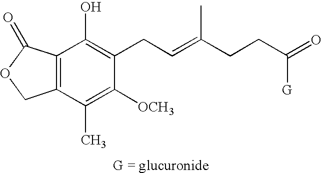

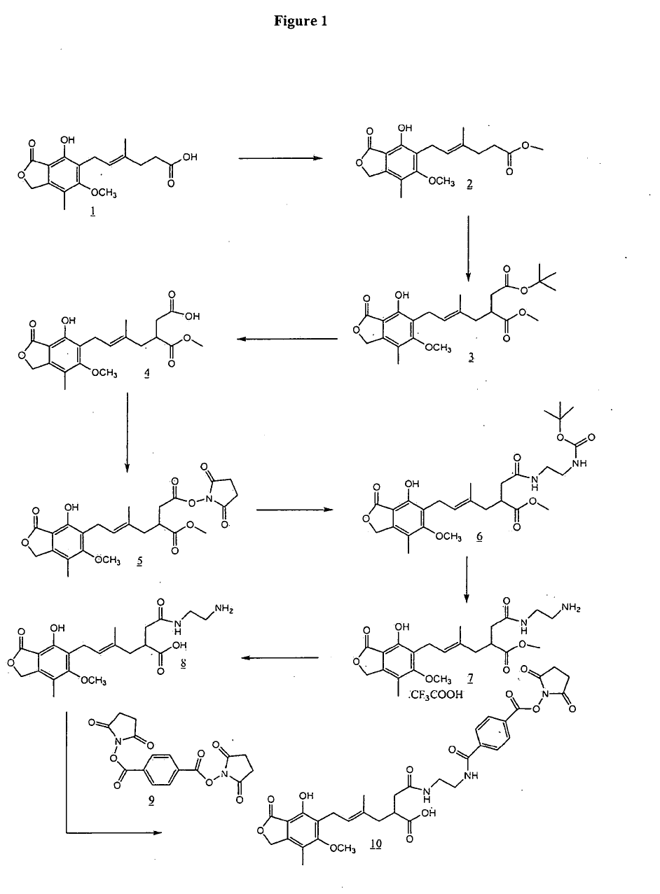

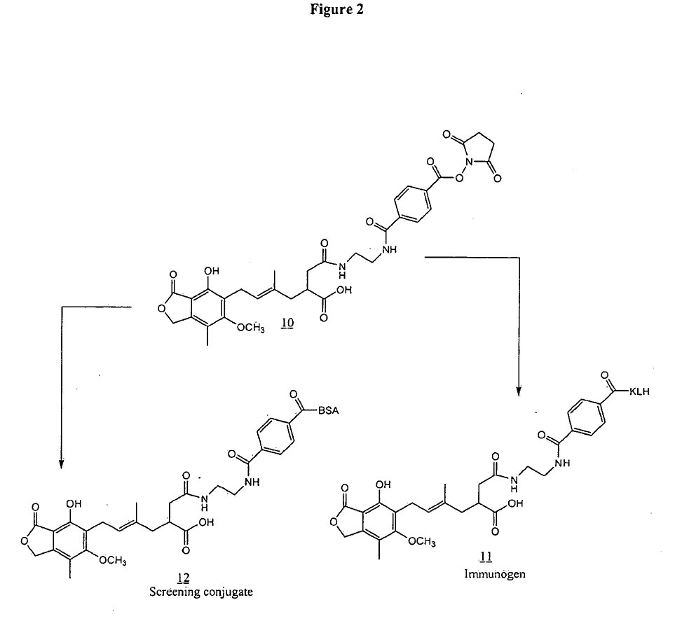

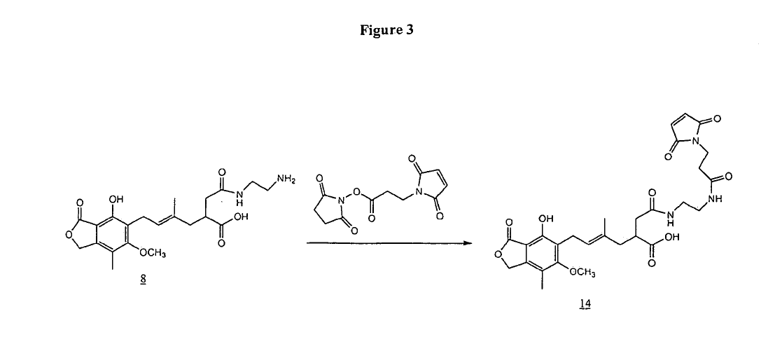

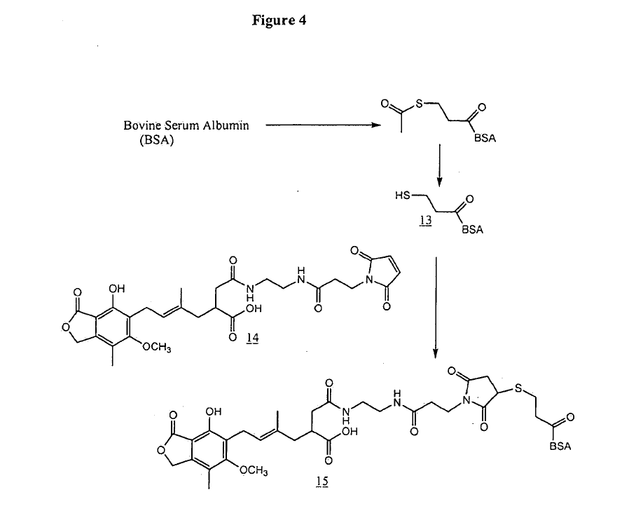

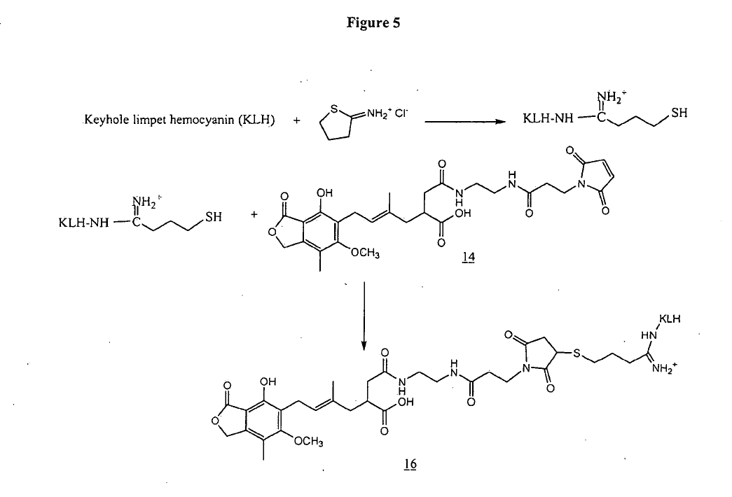

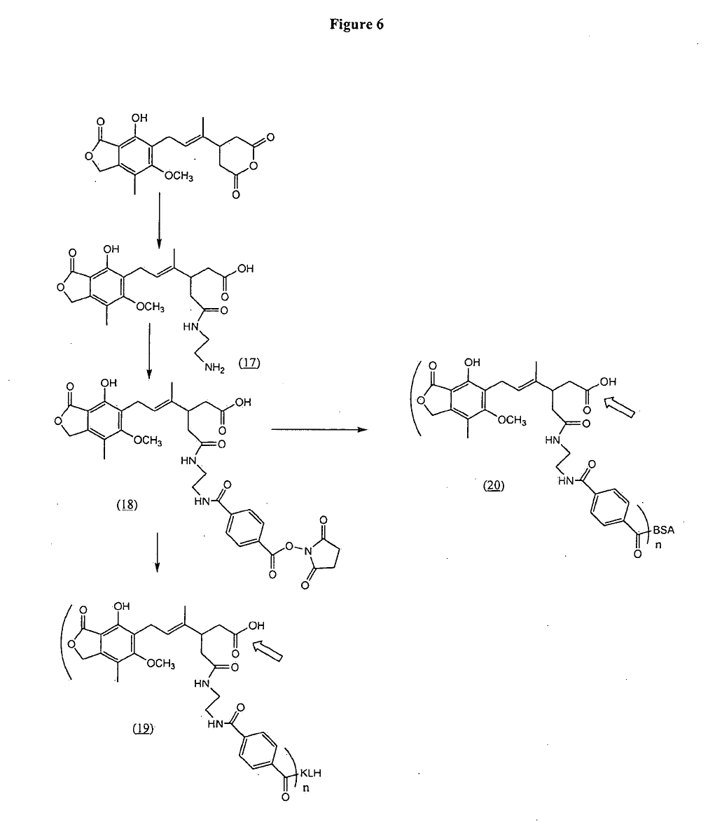

The appended figures depict the preparation of preferred embodiments for mycophenolic acid4′-and 5′-immunogen, respectively, prepared through active ester chemistry (see FIGS. 1, 2 and 6). Another preferred embodiment is the preparation-of immunogens through maleimide/thiol chemistry (see FIGS. 3-5).

Mycophenolic acid conjugated to carrier through a 5′ or 4′-position linker presents the mycophenolic acid molecule optimally to the immune system, including the terminal carboxylic acid. 5′ and 4′ positions of substitution are novel for mycophenolic acid immunogens.

Immunogens in general prepared by derivatization of an alkyl side chain at a non-terminal position are uncommon and for mycophenolic acid in particular would not have been obvious as a solution to the problem of cross-reactive antibodies.

In one embodiment, the invention relates to a compound having the formula

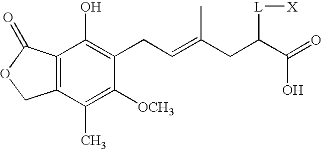

wherein L is a linker comprising from 1 to 40 carbon atoms arranged in a straight chain or a branched chain, saturated or unsaturated, and containing up to two ring structures and 0-20 heteroatoms, with the proviso that not more than two heteroatoms may be linked in sequence and X is selected from the group consisting of polypeptides, polysaccharides, and synthetic polymers.

In another embodiment, the invention relates to an antibody generated in response to the compound immediately above.

In another embodiment, the invention relates to a compound having the formula

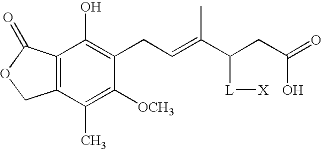

wherein L is a linker comprising from 1 to 40 carbon atoms arranged in a straight chain or a-branched chain, saturated or unsaturated, and containing up to two ring structures and 0-20 heteroatoms, with the proviso that not more than two heteroatoms may be linked in sequence and X is selected from the group consisting of polypeptides, polysaccharides and synthetic polymers.

In another embodiment, the invention relates to an antibody generated in response to the compound immediately above.

Another object of the present invention is to provide antibodies to MPA generated from the immunogens of the invention. In order to generate antibodies, the immunogen can be prepared for injection into a host animal by rehydrating lyophilized immunogen to form a solution or suspension of the immunogen. Alternatively, the immunogen may be used as a previously prepared liquid solution or as a suspension in buffer. The immunogen solution is then combined with an adjuvant such as Freund's to form an immunogen mixture. The immunogen may be administered in a variety of sites, at several doses, one or more times, over many weeks.

Preparation of polyclonal antibodies using the immunogens of the invention may follow any of the conventional techniques known to those skilled in the art. Commonly, a host animal such as a rabbit, goat, mouse, guinea pig, or horse is injected with the immunogen mixture. Further injections are made, with serum being assessed for antibody titer until it is determined that optimal titer has been reached. The host animal is then bled to yield a suitable volume of specific antiserum. Where desirable, purification steps may be taken to remove undesired material such as nonspecific antibodies before the antiserum is considered suitable, for use in performing assays.

Monoclonal antibodies may be obtained by hybridizing mouse lymphocytes, from mice immunized as described above, and myeloma cells using a polyethylene glycol fusion method such as the technique described in Methods in Enzymology 73 (Part B), pp. 3-46, 1981.

In order that the invention may be more readily understood, reference is made to the following examples, which are intended to illustrate the invention, but not limit the scope thereof. In the examples that follow, underlined numbers in boldface type refer to the corresponding structure in the drawings.

### SPECIFIC EMBODIMENTS

### Example 1

### Preparation of Mycophenolic Acid Methyl Ester (2)

To a solution of 5.0 g (15.6 mmol) of mycophcnolic acid in anhydrous methanol was added 100 μL of concentrated H₂SO₄. This clear brownish solution was allowed to stir under argon atmosphere at room temperature for 18 h. The resulting-reaction mixture was concentrated under reduced pressure to give a brownish-white powder. This was dissolved in 150 mL of dichloromethane and was washed with saturated sodium bicarbonate (3×100 mL) followed by 100 mL of water. The dichloromethane layer was collected, dried (anh. Na₂SO4) and was concentrated to give 4.72 g (14.1 mmol) of mycophenolic acid methyl ester (2) as a pale brown powder.

### Example 2

### Preparation of 5′-t-butoxycarbonylmethyl-mycophenolic Acid Methyl Ester (3)

A solution of 26 mL (26 mmol) of sodium bis (trimethylsilyl)amide (1.0 M solution in THF) was cooled in dry ice/acetone bath to −78° C. under argon atmosphere. To this cooled solution was added 2.6 mL (22 mmol) of dimethyl propylene urea (DMPU) and allowed to stir at −78° C. for 15 minutes. A solution of 2.86 g (8.56 mmol) of mycophenolic acid methyl ester (2) in 45 mL of freshly distilled THF was added dropwise to the reaction mixture. The reaction mixture was allowed to stir at −78° C. for 1 hour and the color of the reaction mixture was turried from pale yellow to yellow-orange. To the reaction mixture was added 1.9 mL (912 mmol) of t-butyl bromoacetate and the reaction mixture was allowed to stir at -78° C. for 3 hours. The reaction was quenched with 20 mL of saturated ammonium chloride solution and the resulting mixture was allowed to warm up to room temperature. An additional 200 mL of saturated ammonium chloride solution was added and the reaction mixture was extracted with 3×200 mL of ethyl acetate. The combined organic layer was washed with 300 mL of saturated ammonium chloride, dried (anh. Na₂SO₄), and concentrated. The crude product was purified by silica gel column chromatography using 30% hexane in ethyl acetate to give 3.87 g of a semi solid. A portion of this product (1.45 g) was purified by RP-HPLC [Rainin C-18(ODS) 21.4 mm×250 mm] using a gradient system of acetonitrile/water containing 0.1% of trifluoroacetic acid in several runs. Product containing fractions were combined and acetonitrile was evaporated. The residue was lyophilized to give 669 mg (1.49 mmol, 47%) of 5′-t-butoxycarbonylmethyl-mycophenolic acid methyl ester (3).

### Example 3

### Preparation of 5′-carboxymethyl-mycophenolic Acid Methyl Ester (4)

To 300 mg (0.67 mmol) of 5′-t-butoxycarbonylmethyl-mycophenolic acid methyl ester (3) was added 15 mL of a solution of trifluoroacetic acid in dichloromethane. The mixture was allowed to stir at room temperature for 0.5 hr and concentrated. The residue was purified by silica gel column chromatography using 20% methanol in ethyl acetate to-give 250 mg (0.63 mmol, 95%) of 5′-carboxymethyl-mycophenolic acid methyl ester (4).

### Example 4

### Preparation of 5′-(Ssccinimido-N-oxy)carbonylmethyl-mycophenolic Acid Methyl Ester

To 200 mg (0.51 mmol) 5′-carboxymethyl-mycophenolic acid methyl ester (4) in 2 mL of freshly distilled THF was added 244 mg (2.12 mmol) of N-hydroxysuccinimide and 437 mg (2.12 mmol) of dicyclohexylcarbodiimide. The mixture was allowed to stir at room temperature for 2 h. The precipitated dicyclohexylurea was filtered off and the filtrate was purified using preparative RP-HPLC (Waters Delta Pak C-18 50×250 mm, water/acetonitrile/0.1% TFA).

Product containing fractions were pooled and immediately lyophilized to give 129 mg (52%, 0.26 mmol) of 5′-(succinimido-N-oxy)carbonylmethyl-mycophenolic acid methyl ester (5).

### Example 5

### Preparation of 5′-N-2-t-butoxycarbonyl aminoethyl)amino carbonylmethyl-mycophenolic Acid Methyl Ester (6)

To 680 mg (1.73 mmol) of 5′-carboxymethyl-mycophenolic acid methyl ester (4) was added 75 mL of freshly distilled dichloromethane. To the reaction mixture was added 20 ml solution of 516 mg (3.25 mmol) of t-butyl N-(2-aminoethyl)-carbamate in freshly distilled dichloromethane. The resulting mixture was allowed to stir at room temperature and 503 mg (2.62 mmol) of 1(3-dimethylaminopropyl)-3-ethylcarbodiimide hydrochloride was added. The reaction mixture was allowed to stir at room temperature 18 h. The reaction mixture was washed with 2×100 mL of water, 2×100 mL of saturated sodium bicarbonate followed by 100 mL of water. The organic layer was separated, dried (anh. Na₂SO₄) and concentrated. The residue was purified by silica gel column chromatography using 10% methanol in ethyl acetate to give 472 mg (0.88 mmol, 51%) of 5′-(N-2-t-butoxycarbonyl aminoethyl)aminocarbonylmethyl -mycophenolic acid methyl ester (6) as a colorless gum.

### Example 6

### Alternative Preparation of 5′-(N-2-t-butoxycarbonyl aminoethyl) aminocarbonylimethyl-mycophenolic Acid Methyl Ester (6) from 5′-(succinimido-N-oxy) carbonylmethyl-mycophenolic Acid Methyl Ester (5)

A solution of 125 mg (0.25 mmol) of 5′-(succinimido-N-oxy)carbonylmethyl-mycophenolic acid methyl ester (5) in 5 mL of freshly distilled dichloromethane is prepared. To this reaction mixture a solution of 59 μL (0.38 mmol) of t-butyl-N-(2-aminoethyl)-carbamate in 2 mL of dichloromethane is added. The reaction mixture is allowed to stir at room temperature and 73 μL (5.2 mmol) of triethylamine was added. The reaction mixture is allowed to stir at room temperature 18 h and the resulting reaction mixture is washed with water, saturated sodium bicarbonate followed by water. The organic layer is dried (Na₂SO₄), and concentrated. The residue is purified by silica gel column chromatography to give of 5′-(N-2-t-butoxycarbonyl aminoethyl) aminocarbonylmethyl -mycophenolic acid methyl ester (6).

### Example 7

### 5′-(N-2-aminoethyl)aminocarbonylmethyl-mycophenolic Acid Methyl Ester Trifluoroacetic Acid Salt (2)

To a solution of 55 mg (0.102 mmol) of 5′-(N-2-t-butoxycarbonyl aminoethyl) carbonylmethyl-mycophenolic acid methyl ester (6) in 4 mL of freshly distilled dichloromethane was added 4 mL of trifluoroacetic acid. The resulting mixture was allowed to stir at room temperature for 15 minutes and concentrated under reduced pressure to give 56 mg (0.102 mmol,) of 5′-(N-2-aminoethyl) aminocarbonylmethyl -mycophenolic acid methyl ester trifluoroacetic acid salt (U in quantitative yield as a pale yellow gum.

### Example 8

### 5′-(N-2-aminoethyl)aminocarbonylmethyl-mycophenolic Acid (8)

To 1.63 g (2.97 mmol) of 5′-(N-2-aminoethyl) aminocarbonylmethyl -mycophenolic acid methyl ester trifluoroacetic acid salt (7) is added 22 mL of THF, 22 mL of methanol and 3.5g (83 mmol) of lithium hydroxide monohydrate in 48 mL of water. The reaction mixture is allowed to stir at room temperature 18 h and concentrated to remove THF and methanol as much as possible. To the resulting reaction mixture phosphoric acid is added to adjust the pH to 5 and the aqueous -reaction mixture is extracted with 6×50 mL of chloroform. The organic layers are combined, dried (Na₂SO₄) and concentrated to give 5′-(N-2-aminoethyl) aminocarbonylmethyl-mycophenolic acid (8).

### Example 9

### Preparation of Terephthalic Acid Bis-(2,5-dioxo-pyrrolidin-1-yl)ester (9)

To 15 g (73.8 mmol) of terephthaloyl chloride was added 300 mL of dichloromethane. The mixture was allowed to stir at 0C for 10 minutes under argon atmosphere. To the reaction mixture was added 30 g(0.26 mol) of N-hydroxysuccinimide followed by 30 mL (0.22 mol) of triethylamine dropwise at 0° C. The mixture was allowed to warm up at room temperature for 2 days. The solid was filtered off and the residue was washed with 200 mL of dichloromethane. The residue was resuspended in 300 mL of dichloromethane and allowed to stir for 10 minutes. The solid was filtered and dried to give 24.1 g (66 mmol) of (9) as a white powder.

### Example 10

### Mycophenolic Acid 5′-Substituted Aromatic NHS Ester, N-{2-[(E)-3-carboxy-7-(4-hydroxy-6-methoxy-7-methyl-3-oxo-1,3-dihydro-isobenzofuran-5-yl)-5-methyl-hept-5-enoylamino]-ethyl}-terephthalamic acid 2,5-dioxo-pyrrolidin-1-yl Ester (10)

A solution of 500 mg (1.18 mmol) of mycophenolic acid amine (8) in 15 mL of anhydrous DMF is made at room temperature. To this magnetically stirred solution, 360 μL (3.5 mmol) of triethylamine is added. This solution is added dropwise to a solution of 428 mg (1.18 mmol) of terephthalic acid di NHS ester in 10 mL of freshly distilled THF. The reaction mixture is allowed to stir at room temperature 18 h under argon atmosphere and concentrated under reduced pressure. The crude product is purified by RP-preparative HPLC using a gradient run consisting of acetonitrile-water containing 0.1% TFA. All fractions containing product are lyophilized to give the desired NHS ester (10).

### Example 11

### Preparation of Mycophenolic Acid Immunogen (11)

To 180 mg of keyhole limpet hemocyanin (KLH, Pierce chemical) in 7 ml of 50 mM potassium phosphate (pH 7.5) is cooled in an ice-bath. To the solution is added 10.5 mL of dimethylsulfoxide (DMSO) dropwise and the reaction temperature is maintained below room temperature. To the protein solution is added a solution of 54 mg (0.08 mmol) of ( to in 1.5 mL of DMF dropwise. The mixture is allowed to stir at room temperature 18 h. The resulting conjugate is placed in a dialysis tube (10,000 MW cut-off) and is dialyzed in 1 L of 70% DMSO in 50 mM potassium phosphate (pH 7.5, 3 changes, at least 3 hours each), 1 L of 50% DMSO in 50 mM potassium phosphate (at least 3 hours), 1 L of 30% DMSO in 50 mM potassium phosphate (at least 3 h), 1 L of 10% DMSO in 50 mM potassium phosphate (at least 3 h) at room temperature, followed by 6 changes with 50 mM potassium phosphate (pH 7.5) at 4° C. (1 L each for at least 6 h each). The protein concentration is determined by using BioRad Coomassie blue protein assay (Bradford, M., Anal. Biochem. 72,248,1976). The extent of available lysine modification is determined by the TNBS method (Habeeb AFSA, Anal. Biochem.14, 328-34,1988).

### Example 12

### Preparation of Mycophenolic Acid BSA Conjugate (12)

A solution of 800 mg of bovine serum albumin (BSA) in 8 mL of 50 mM potassium phosphate (pH 7.5) is cooled in an ice-bath. To the solution is added 12 mL of DMSO dropwise and the reaction mixture is maintained below room temperature. To the reaction mixture a solution of 20 mg (0.030 mmol) of mycophenolic acid derivative (1) in 1 mL of anh. DMF is added dropwise. This is allowed to stir at room temperature 48 h, the resulting conjugate is placed in a dialysis tube (10,000 MW cut-off) and is dialyzed in 1 L of 70% DMSO in 50 mM potassium phosphate (pH 7.5, 3 changes, at least 3 hours each), 1 L of 50% DMSO in 50 mM potassium phosphate (at least 3 hours), 1 L of 30% DMSO in 50 mM potassium phosphate (at least 3 h), 1 L of 10% DMSO in 50 mM potassium phosphate (at least 3 h) at room temperature, followed by 6 changes with 50 mM potassium phosphate (pH 7.5) at 4° C. (1 L each for at least 6 h). The protein concentration is determined using BioRad Coomassie blue protein assay (Bradford, M., Anal. Biochem. 72,248,1976).

### Example 13

### Preparation of Maleimido Linked Mycophenolic Acid (E)-2-({2-[3-(2,5-dioxo-2,5-dihydro-pyrro-1-1-yl)-propionylamino]-ethylcarbamoyl}-methyl)-6-(4-hydroxy-6-methoxy-7-methyl-3-oxo-1,3-dihydro-isobenzofuran-5-yl)-4-methyl-hex-4-enoic acid (14)

A solution of 200 mg (0.47 mmol) of mycophenolic acid amine (8) in 8 mL of anhydrous DMF is prepared. To this solution 165 μL (1.18 mmol) of triethylamine is added. This solution is added dropwise to a solution of 139 mg (0.52 mmol) of 3-maleimido-propionic acid N-hydroxysuccinimide ester in 8 mL of freshly distilled THF. The reaction mixture is allowed to stir at room temperature 18 h under argon atmosphere and concentrated under-reduced pressure. The crude product is purified by silica gel column chromatography to give the desired maleimido linked mycophenolic acid (14).

### Example 14

### Mycophenolic Acid-Succinimidyl-Sulfanylpropionyl-BSA Conjugate (15)

Bovine serum albumin (0.5 g) is dissolved in 50 mL of 50 mM potassium phosphate containing 1 mM EDTA. To the reaction mixture 1.24 mL of N-succinimidyl S-acetylthiopropionate (SATP) in DMSQ (15 mg/mL in DMSO) is added. The reaction mixture is allowed to stand at room temperature for 1 h. The resulting solution is dialyzed against 50 mM potassium phosphate (pH 7.5) [10,000 MW cut-off] over a period of 3 days to give a solution of BSA-SATP which is stored at −20° C. for future use. The protein concentration is determined using BioRad Coomassie blue protein assay (Bradford, M., Anal. Biochem. 72:248,1976).

To 5 mL of BSA-SATP (9 mg/mL) is added 850 μL of the following buffer; 50 mM potassium phosphate, 25 mM EDTA, 0.5 M NH₂OH, pH 7.2. The mixture is vortexed and allowed to stand at room temperature for 2 h to deprotect the S-acetyl moiety.

The resulting solution is desalted using 3 PD 10 columns to produce 5.5 ml of protein solution containing the corresponding deprotected thiopropionyl-BSA. This solution is cooled to 0° C. and 4 mL of DMSO is added dropwise. A solution of 10 mg (0.017 mmol) of mycophenolic acid-maleimido derivative (14) in 0.5 mL of DMSO is added to the protein solution. The mixture is allowed to stir at room temperature for 24 h. To the protein solution 400 μL of 5 mg/mL ethylmaleimide in DMSO is added and allowed to stir at-room temperature for 24 h. The resulting conjugate is placed in a dialysis tube (10,000 MW cut-off) and dialyzed in 1 L of 30% DMSO in 50 mM potassium phosphate (pH 7.5, 3 changes, at least-3 hours each), 1 L of 20% DMSO in 50 mM potassium phosphate (at least 3 hours), 1 L of 10% DMSO in 50 mM potassium phosphate (at least 3 h), followed by 6 changes with 50 mM potassium phosphate (pH 7.5) at 4° C. (1 L each for at least 6 h each). The protein concentration is determined using Biorad Coomassie blue protein assay (Bradford, M., and Anal; Biochem. 72:248, 1976).

### Example 15

### Mycophenolic Acid Succinimidyl-Sulfanylbutyrimidoyl-KLH (16)

Keyhole limpet hemocyanin (KLH, 60 mg, Pierce chemicals) is reconstituted in 100 mM sodium phosphate buffer at pH 7.2. 2-Iminothiolane (2-IT, 13.5 mg) is added to the protein solution as a solid and the reaction is allowed to stir-at room temperature in the dark and under argon atmosphere for 1 h. The thiolated KLH is desalted on a Sephadex PD-10 column pre-equilibrated with 100 mM sodium phosphate buffer at pH 6.5. The thiol loading is determined per KLH molecule. (MW 5,000,000). To 6 ml of KLH-(SH)ₙ [4.7 mg/mL] is added a solution of 21 mg (036 mmol) of mycophenolic acid maleimide (14) in 1 mL of DMF dropwise and the mixture is allowed to stir at room temperature 18 h. The resulting conjugate is placed in a dialysis lube (10,000 MW cut-off) and is dialyzed in 1 L of PBS buffer (180 mM NaCl, 10 mM sodium phosphate, pH 7.21 containing 20% DMF. (3 times at least 6 h each). This is followed by 1 L of PBS buffer, pH 7.2 at 4° C. to give a solution of the KLH conjugate (16). The protein concentration is determined by using BioRad Coomassie blue protein assay (Bradford, M., Anal. Biochem. 72:248,1976).

### Example 16

### 4′-(N-2-aminoethyl)-aminocarbonyl-MPA (17)

4′-Carboxymethyl-MPA (synthesized as described in U.S. Pat. No. 6,811,998 B2, Example 43), 41 mg (0.1084 mmol), is dissolved in 0.82 ml of THF, 8.77 μl of pyridine and 15.3 μl of trifluoroacetic anhydride is added. The mixture is stirred for 3 hr, then 0.1084 mmol of ethylenediamine in 820 μl of pyridine is added. The mixture is allowed to stir at room temperature for 16 hr before purifying by RP-HPLC in a gradient of acetonitrile in 0.1 % aqueous TFA to give product as the trifluoroacetic acid salt.

### Example 17

### 4′-(2,5-dioxo-pyrrolidinyl-1-yl-oxocarbonyl-4″-phenyl-1″-carbonylamino-ethyl-2-aminocarbonylmethyl)-MPA (18)

A solution of 630 mg (1.18 mmol) of 4′-(N-2-aminoethyl)-aminocarbonyl-MPA trifluoroacetic acid salt in 15 mL of anhydrous DMF is prepared at room temperature. To this magnetically stirred solution, 360 μL (3.5 mmol) of triethylanine is added. This solution is added-dropwise to a solution of 428 mg (1.18 mmol) of terephthalic acid diNHS ester in 10 mL of freshly distilled THF. The reaction mixture is allowed to stir at room temperature 18 h under argon atmosphere and concentrated under reduced pressure. The crude product is purified by RP-preparative HPLC using a gradient run consisting of acetonitrile- water containing 0.1% TFA. All fractions containing product are immediately frozen and lyophilized to give the desired NHS ester.

### Example 18

### MPA-4′-KLH Immunogen (19)

The KLH immunogen is prepared in a similar manner as for the 5′-immunogen (11), substituting the 4′-activated hapten (18) above for the 5′-activated hapten (10).

### Example 19

### MPA-4′-BSA Conjugate (20)

The BSA conjugate is prepared in a similar manner as for the 5′-conjugate (2), substituting the 4′-activated hapten (18) above for the 5′-activated hapten (10).

Having described the invention in detail and by reference to specific embodiments thereof, it will be apparent that modifications and variations are possible without departing from the scope of the invention defined in the, appended claims. More specifically, although some aspects of the present invention are identified herein as preferred or particularly advantageous, it is contemplated that the present invention is not necessarily limited to these preferred aspects of the invention.

What is claimed is:

1. A compound having the formula 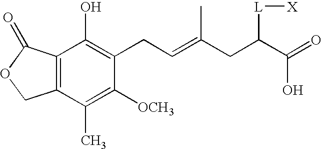
wherein L is a linker comprising from 1 to 40 carbon atoms arranged in a straight chain or a branched chain, saturated or unsaturated, and containing, up to two ring structures and 0-20 heteroatoms, with the proviso that not more than two heteroatoms may be linked in sequence and X is selected from the group consisting of polypeptides, polysaccharides, and synthetic polymers.

2. An antibody generated in response to the compound of claim 1.

3. A compound having the formula 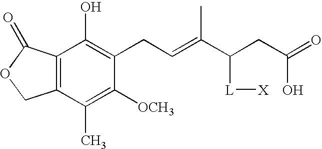
wherein L is a linker comprising from 1 to 40 carbon atoms arranged in a straight chain or a branched chain, saturated or unsaturated, and containing up to two ring structures and 0-20 heteroatoms, with the proviso that not more than two heteroatoms may be linked in sequence and X is selected from the group consisting of polypeptides, polysaccharides and synthetic polymers.

4. An antibody generated in response to the compound of claim 3.
